(19) United States
(12) Patent Application Publication
(10) Pub. No.: US 2025/0164388 A1
(43) Pub. Date: May 22, 2025

(54) Method and device for non-invasive tomographic characterisation of a sample comprising a plurality of differentiated tissues

(71) Applicants: INESC TEC - Instituto de Engenharia de Sistemas e Computadores Tecnologia e Ciência, Porto (PT); Universidade do Porto, Porto (PT)

(72) Inventors: Rui Miguel DA COSTA MARTINS, Porto (PT); Filipe BAPTISTA NEVES DOS SANTOS, Porto (PT); Mário Manuel DE MIRANDA FURTADO CAMPOS CUNHA, Porto (PT); Aníbal Filipe MARQUES MONTEIRO DA SILVA, Porto (PT); Renan TOSIN, Porto (PT); Sandro Augusto COSTA MAGALHÃES, Porto (PT); Mafalda REIS PEREIRA, Porto (PT)

(21) Appl. No.: 18/724,833

(22) PCT Filed: Dec. 30, 2022
(86) PCT No.: PCT/EP2022/088102

§ 371 Date: Jun. 27, 2024

(30) Foreign Application Priority Data
Dec. 31, 2021 (PT) 117727
Jan. 5, 2022 (PT) 117732

(51) Int. Cl. G01N 21/27; G01N 33/02

(52) U.S. Cl. CPC G01N 21/27; G01N 33/025

(57) ABSTRACT

The present document discloses a method and device for a non-invasive tomographic characterization of a sample comprising a plurality of differentiated tissues. In particular, a computer-implemented method for non-invasive tomographic metabolite characterization of a vegetable or animal sample to be characterized, the sample comprising a plurality of differentiated tissues from a set of previously-characterized differentiated tissues, by using a multidimensional latent structure model of differentiated tissues.

### TECHNICAL FIELD

The present disclosure relates to a method and device for non-invasive tomographic characterisation of a sample comprising a plurality of differentiated tissues.

### BACKGROUND

Mainstream analytical chemistry and biosensor approaches to spectroscopy information complexity are to use physical separation unit operations [1,2], reaction-specific labelling [3,4], or more recently label-free reaction specificity [5]. State-of-the-art chemometrics makes use of macroscopic hyperspectral or multispectral imaging to relate surface characteristics of plant tissues (e.g., fruits or leaves) to pigmentation and disease symptoms [6]. The previous art methods are cumbersome to be used in vivo, particularly in an open field context:
i. Biosensors need destructive sampling, consumables, provide limited sampling or even need sample preparation, all of which make them impractical to be implemented on robotic platforms for high-throughput monitoring of vast agricultural areas;
ii. Hyperspectral cameras capture a picture in a wavelength interval of light reflected from the plant surface. The reflected light is non-absorbed light; therefore, it carries little information about the sample composition, with insufficient information relating to omics quantitative information.

These facts are disclosed in order to illustrate the technical problem addressed by the present disclosure.

### GENERAL DESCRIPTION

The present document discloses a computer-implemented method for non-invasive tomographic metabolite characterisation of a vegetable or animal sample to be characterised, the sample comprising a plurality of differentiated tissues from a set of previously-characterised differentiated tissues, by using a multidimensional latent structure model of differentiated tissues, which was previously obtained by: scanning tissue samples belonging to each differentiated tissue of the set with a hyperspectral microscope and spectrometer; acquiring hyperspectral spectrum or spectra for each differentiated tissue sample from the hyperspectral microscope and spectrometer; analysing the same tissue samples for the metabolites to be characterised; calculating the latent structure model from the acquired spectra and analysed metabolites; the method comprising the steps of: spatially scanning the sample to be characterised with a hyperspectral point-of-measurement, POM, optical device and a spectrometer; acquiring hyperspectral spectrum or spectra from each of a plurality of spatial positions and/or orientations of the POM optical device and spectrometer in respect of the sample to be characterised; using the previously obtained model to deconvolute the metabolites to be characterised for each of the differentiated tissues from the acquired spectra.

In an embodiment, the scanning of tissue samples belonging to each tissue of the set with a hyperspectral microscope and spectrometer is microscopic, and the spatially scanning the sample to be characterised with a hyperspectral POM optical device and spectrometer is macroscopic.

In an embodiment, the spatially scanning comprises varying one or more of the following to obtain a spatial diversity of the POM spatial scanning: displacement of the POM optical device; point-of-view angle of the POM optical device; different scales of magnification of the POM optical device; moving the POM optical device lines of sight to cause a parallax effect.

In an embodiment, the multidimensional latent structure model is a hierarchical latent structure model.

In an embodiment, the hierarchical latent structure model comprises a latent variable model comprising a latent variable super-space and corresponding latent variable sub-spaces.

In an embodiment, the hierarchical relationship of the hierarchical latent structure model comprises super-space and corresponding sub-spaces: a latent sub-space for spectral data of each of the differentiated tissues; a super-space for each of the metabolites to be characterised, translating the combination of hyperspectral data to the metabolites to be characterised.

In an embodiment, the sample to be characterised is a three-dimensional tissue structure or organ.

In an embodiment, the metabolites to be characterised comprise pigments, tannins, photosynthesis, energy storage, nutrient uptake, necrosis, infection probability, or combinations thereof.

In an embodiment, the sample to be characterised is a vegetable sample, in particular the sample to be characterised is a tomato or grape tissue sample.

In an embodiment, the sample to be characterised is vegetable sample and the differentiated tissues comprise skin, pulp, and seed tissue.

In an embodiment, the scanning of tissue samples belonging to each differentiated tissue is carried out along a sampling grid.

In an embodiment, the scanning of tissue samples belonging to each differentiated tissue is further carried out along discrete sampling depths.

In an embodiment, the spectra are UV-VIS-NIR.

It is further disclosed a device for non-invasive tomographic metabolite characterisation of a vegetable or animal sample to be characterised, the sample comprising a plurality of differentiated tissues from a set of previously-characterised differentiated tissues, including a computer processor and a non-transitory computer-readable medium comprising a multidimensional latent structure model of differentiated tissues, which was previously obtained by: scanning tissue samples belonging to each differentiated tissue of the set with a hyperspectral microscope and spectrometer; acquiring hyperspectral spectrum or spectra for differentiated each tissue sample from the microscope and spectrometer; analysing the same tissue samples for the metabolites to be characterised; calculating the latent structure model from the acquired spectra and analysed metabolites; wherein the computer processor is configured for: spatially scanning the sample to be characterised with a hyperspectral point-of-measurement, POM, optical device and spectrometer; acquiring hyperspectral spectra from a plurality of spatial positions and/or orientations of the POM optical device and spectrometer in respect of the sample to be characterised; using the previously obtained model to deconvolute the metabolites to be characterised for each of the differentiated tissues from the acquired spectra.

It is also presented a non-transitory computer-readable medium comprising computer program instructions for implementing a device for non-invasive tomographic metabolite characterisation of a vegetable or animal sample to be characterised, which when executed by a processor, cause the processor to carry out the disclosed method.

One of the present disclosure aims is to develop a non-invasive spectroscopy tomography system for compositional imaging of plant and animal tissues. This technology is demonstrated in application to precision agriculture and plant physiology. The present disclosure is based on image reconstruction using latent hierarchical information fusion methods to unscramble the observed spectroscopy signal into its different components of the observed tridimensional structures, particularly the different plant tissues being recorded, such as in grapes the skin, pulp, and seeds. Hierarchical relations exploit the parallax effect, which changes the spectral fingerprint depending upon the point-of-view angle of the spectroscopy probe. Reconstruction is performed by the hierarchical latent relationships between spectral patterns of the observed tissues. This principle is used to develop:
i. macroscopic tomography—for measuring macroscopic properties of plant internal tissues with case studies of grape and tomato fruits (e.g., skin, pulp, and seeds);
ii. microscopic tomography—for measuring microscopic spectral patterns that can provide a diagnosis at very early stages, e.g., of tomato leaf bacterial infection by Pseudomonas syringae and Xanthomonas euvesicatoria; and
iii. Multi-scaled tomography results-exploiting the hierarchical parallax relationship between latent spaces of different scales of magnification to provide microscopic reconstructions based on macroscopic measurements.

Results show the feasibility of relationship reconstruction from latent structures, as well as, providing relationship between macroscopic and microscopic spectral data.

Spectral data carries both physical and chemical information. The observed spectrum of plant tissue is the superposition of all structures in the field of view, scattering, matrix effects, and multi-scaled interference. Being able to use these complex interactions is paramount to unscrambling the information present in each recorded spectrum. The complexity of spectral data has been the foremost hurdle to the existence of in vivo, reagent-less, and non-invasive chemical or metabolic diagnosis.

The macroscopic spectra are recorded and related to the hyperspectral image by their corresponding latent space.

The capacity of internal structures reconstruction, compositional, and disease diagnosis at very early stages centres molecular biology and plant physiology as the main driver of information in precision agriculture and plant biotechnology. The present disclosure bridges the information between in vitro laboratory and in vivo field studies by allowing high-throughput and multi-scaled monitoring of plants in their ecosystem without disrupting the interaction of plants with soil, climate, and ecosystem. Allowing information transfer between the field and laboratory tests is a critical enabling transformation in precision agriculture that allows evaluating to be much faster, thus promoting scientific advances in both phytopharmaceuticals and agricultural practices for early-stage and low-impact practices.

One of the main objectives of the disclosure is to provide a macroscopic, microscopic, and multi-scaled spectral reconstruction, compositional, and infection diagnosis through spectroscopy POM devices and advanced signal/information processing, demonstrating the feasibility of tomographic reconstruction by hierarchical latent structures methods.

### BRIEF DESCRIPTION OF THE DRAWINGS

The following figures provide preferred embodiments for illustrating the disclosure. They should not be seen as limiting the scope of the invention.

FIG. 1: Schematic representation of an embodiment of a device, robotic platform and metabolic quantification according to the disclosure.

FIG. 2: Schematic representation of an embodiment of 3D internal spectroscopy reconstruction method using hierarchical latent spaces.

FIG. 3: Schematic representation of an embodiment of hyperspectral microscopy tomography and relationship to macroscopic measurements: (a) hyperspectral image data of infected leaf along different depths (z) where the extension of the infected lesions is explicit in spectral patterns; and (b) macroscopic measurements with device and corresponding classification of infection using linear discriminant analysis (LDA) to spectral data.

FIG. 4: Schematic representation of an embodiment of multi-scaled hyperspectral tomography reconstruction of latent structures equivalence of information as the macroscopic information is the superposition and convolution of the microscopic information, resulting to less abrupt changes in the macroscopic spectra that once related to the micro-scale information open the possibility of non-invasive diagnosis by simple optical adaptations to existing hardware.

FIG. 5: Schematic representation of an embodiment of macroscopical setup consisting of a manual probe to acquire data on select matrix spots.

FIG. 6: Schematic representation of an embodiment of hyperspectral microscope setup composed by a Zeiss Axiovert.A1 10, connected to an Ocean Insight HR4000 spectrometer 30 via an UV-Vis-NIR 200 μm (I.D.) optical fiber 20, fine-tuned for accurate, simultaneous spectral acquisition of a sampled micrograph.

FIG. 7: Schematic representation of an embodiment of macroscopic tomography in grapes: measurements made with a robotic device and HR4000 spectrometer to the hole grape; correspondence between spectral variance space of the robotic device and HR4000 spectrometer and its relationship to the tomography fusion space, obtained by using the information from skin, pulp and seed variance sub-spaces.

FIG. 8: Schematic representation of an embodiment of macroscopic tomography reconstruction: ‘in-vivo’ measurement of two different grapes at veraison and near harvest time, corresponding ‘in-silico’ reconstruction of the skin, pulp and seed tissues spectra and estimated relevant properties of each tissue.

FIG. 9: Schematic representation of an embodiment of macroscopic results: visual inspection to control and inoculated leaves with Pseudomonas syringae and Xanthomonas euvesicatoria infection progression (left), and corresponding spectral classification using linear discriminant analysis from 24 h to 144 h after inoculation (right).

FIG. 10: Schematic representation of an embodiment of microscopic results: observed spectra at 144 h at the hyperspectral microscope (200×), principal component analysis of spectral signal and infection classification, microscope images and corresponding hyperspectral analysis by variance imaging and the corresponding probability of infection by partial least squares regression discriminant analysis.

FIG. 11: Schematic representation of an embodiment of colony PCR analysis of infected tomato leaves with DNA markers XV14 (713 bp) for detection of Xanthomonas euvesicatoria LMG 905. C−—Colony PCR using distilled water as template. 1, 2, 3—DNA samples from different bacterial colonies were obtained through pathogen isolation assay performed 24 h after infection. 4, 5—DNA samples from different bacterial colonies obtained through pathogen isolation assay performed 48 h after infection. C+—Xanthomonas euvesicatoria DNA stored at −80° C. belonging to the laboratory bacterial collection.

FIG. 12: Schematic representation of an embodiment of colony PCR analysis of infected tomato leaves with DNA markers PST2 (200 bp) for detection of Pseudomonas syringae pv. tomato. C−—Colony PCR using distilled water as template. 1-6—DNA samples from different bacterial colonies were obtained through a pathogen isolation assay performed 24 h after infection. 7-13—DNA samples from different bacterial colonies were obtained through a pathogen isolation assay performed 48 h after infection. C+—Pseudomonas syringae pv. tomato DNA stored at −80° C. belonging to the laboratory bacterial collection.

### DETAILED DESCRIPTION

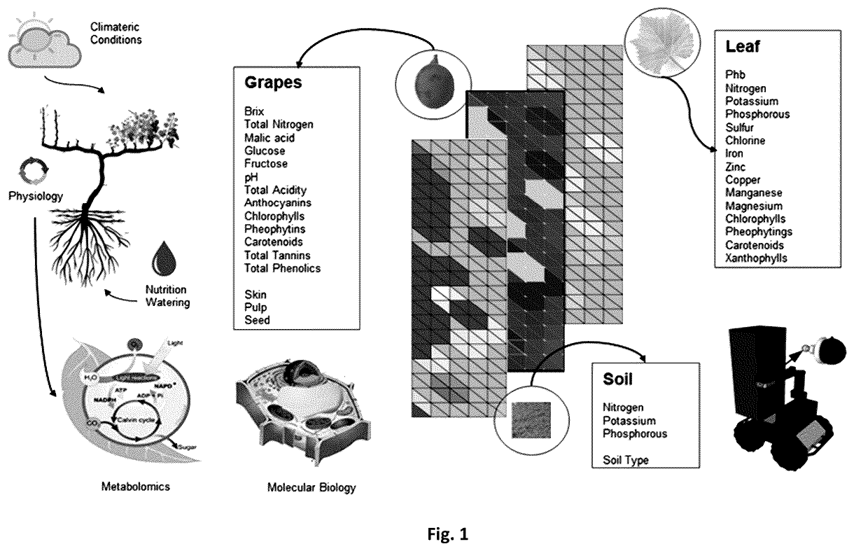

FIG. 1 presents a device, robotic platform and metabolic quantification according to the disclosure. The device comprises a spectroscopy Point-of-Measurement (POM) to record the spectral fingerprint of plant tissues. The POM can record the spectra of the internal structures of plant tissues, allowing the measurement of key metabolites representative of biochemical cycles and regulatory mechanisms, such as pigments, tannins, photosynthesis, energy storage, and nutrient uptake.

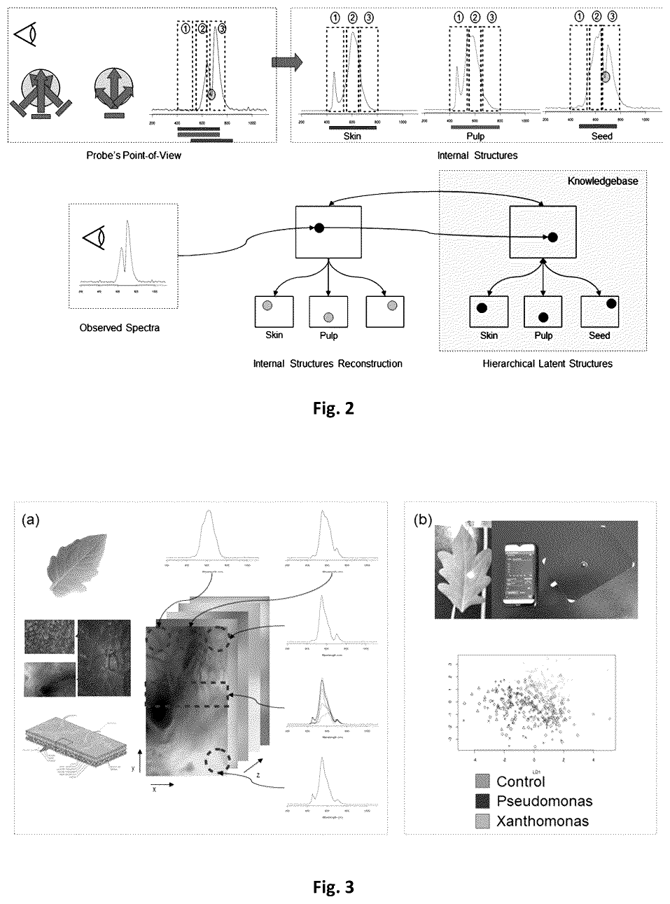

In an embodiment, the present disclosure is deployed/integrated on existing systems and devices, thus extending their technical capabilities. It enables the existing system to explore in detail plant internal structures and composition for a new omics approach to precision agriculture, where molecular biology and plant physiology are key enablers of new diagnosis and agricultural practices. The disclosure includes, FIG. 2. the basic principles for macroscopic tomography reconstruction using latent variables. The method is based in two parts: i. spectral recording of samples at different point-of-view; and ii. establishment of internal structures spectral database.

In an embodiment, the two are related by hierarchical latent structures models in FIG. 2, whereby the information of the object's different perspectives are matched against the knowledge base of tissues to provide a reconstruction of internal sample structures from the observed spectra.

A similar strategy is used for hyperspectral microscopy, as exemplified in FIG. 3.

FIG. 3 presents an embodiment of hyperspectral microscopy tomography and its relationship to macroscopic measurements: (a) hyperspectral image data of infected leaf along different depths (z) where the extension of the infected lesions is explicit in spectral patterns; and (b) macroscopic measurements with device and corresponding classification of infection using linear discriminant analysis (LDA) to spectral data.

In an embodiment, a scanning hyperspectral microscope was implemented to scan at 200× magnification inoculated tomato leaves, taking the spectra along the x, y and z axis. The 3D dataset is stored in the tensor format, to each space coordinate has a corresponding spectrum. The evolution of spectral fingerprints of infection and control are used to create classification models to be used in both hyperspectral microscopy diagnosis and multi-scaled diagnosis.

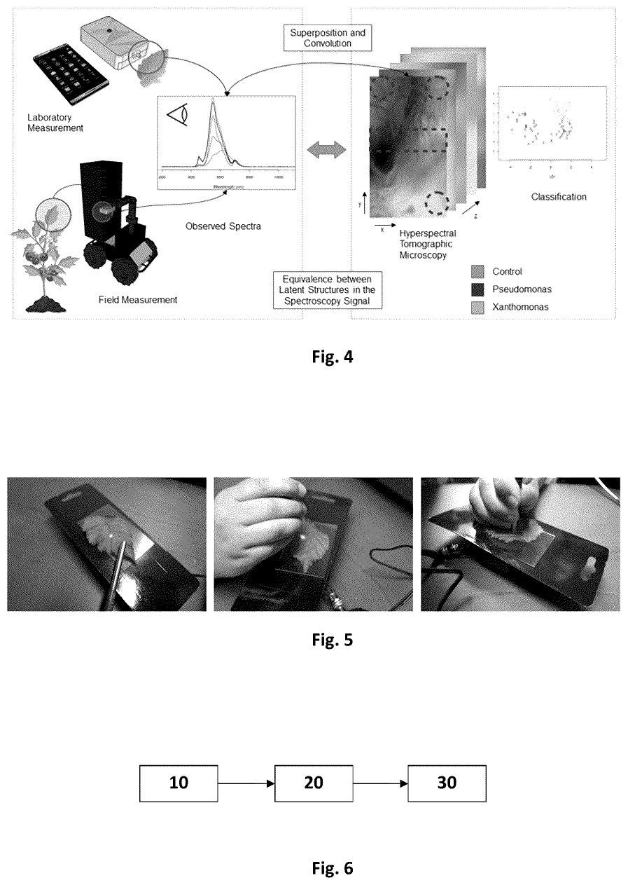

Multi-scaled reconstruction is presented in FIG. 4. In an embodiment, the macroscopic spectra are recorded using 200 μm fiber optics and are related to the 200× hyperspectral image by their corresponding latent space. The latent space correspondence provides the correspondence between the two spectral pieces of information, as the macroscopic record is a superposition of the microscopic hyperspectral images at each z-plane. In this sense, small changes in the macroscopic spectra can be related to the significant changes in the spectra observed with magnification at the different stages of the disease, enabling an early diagnosis with existing low-cost hardware.

Tomato (Solanum lycopersicum L.) plants of the cultivar Cherry were grown in 200 ml pots containing a commercial potting substrate, in a walk-in plant growth chamber under controlled conditions (t=25-27° C., humidity≈60%, and photoperiod of 12/12 h). Plants were divided into three groups: control, Pst and Xeu; control was used as the control (submitted to sterile water treatment), Pst was the group for inoculation with Pseudomonas syringae pv. tomato, whereas Xeu was the group for inoculation with Xanthomonas euvesicatoria LMG 905. Plants were inoculated in the laboratory, at the growth stage of 5-6 fully expanded leaves, by liquid aerosolisation until saturation was achieved with subsequent run-off. Differently inoculated plants were segregated from each other, in order to avoid any possible cross-contamination.

The bacterial suspensions used for these inoculation assays consisted of 1×10⁸ cells·mL⁻¹ for Pst and 2×10⁸ cells·mL⁻¹ for Xeu, prepared from a 48 h old culture, grown on KB medium (peptone, 20.0 g; glycerol, 10.0 mL; MgSO₄, 1.5 g; K₂PO₄, 1.5 g; agar, 15.0 g; distilled water, up to 1.0 liter), for Pst, and YDC medium, for Xeu (yeast extract, 10.0 g; dextrose, 20.0 g; CaCO₃, 20.0 g; agar, 15.0 g; distilled water, up to 1.0 liter). Afterwards, the inoculated plants were covered with transparent polythene bags for 48 h in order to increase the relative humidity that fosters bacterial entry into plant tissues, through natural openings, i.e., stomata.

Plants were monitored daily for symptomatic evolution for 7 days. Leaf samples of all groups (control, Pst and Xeu) were collected once every 24 h, until the 4th day, and one final sampling moment at 144 h, in a total of 5 sampling moments per group.

Simultaneously and in order to verify if the bacteria cultures used in the inoculation assays were viable, 20 μL of Pst and Xeu solution was cultured in Petri dishes containing KB and YDC media, respectively. After 48 h, it was possible to observe bacterial growth in both nutrient media, supporting the bacterial viability at inoculation.

After the inoculation assays, plant leaves from each group were collected and stored in separate plastic bags. Afterwards, for sample disinfection, the surface of each sample was washed in 70% alcohol for 5 min, followed by rinsing with sterile distilled water for 1 min; this procedure was repeated 5 times. Disinfected samples were placed in different homogenising bags containing a side filter. The samples were grounded in 2 ml of sterile distilled water until full tissue maceration was achieved. The obtained mixture was diluted with sterile distilled water to a final volume of 6 mL and collected after passing the filter bag. Twenty microliters of each sample solution were plated in separate Petri dishes containing KB (Pst extracts) and YDC medium (Xeu extracts), respectively, and incubated for 48 h at 28° C. Bacterial colonies that presented characteristic Pst and Xeu morphology were isolated and used in colony PCR.

Primer pairs designed by Vieira et al. [7] were selected for PCR validation of leaves infected with Pst. Similarly, infection of leaves infected with Xeu was confirmed by PCR using primer pairs designed by Albuquerque et al. [8]. Polymerase chain reactions were carried out in 20 μL reactions containing 1× DreamTaq Buffer (Thermo Scientific, Vilnius, Lithuania), 0.2 mM of each dNTP (Thermo Scientific), 0.2 UM of each primer (STAB Vida, Lisbon, Portugal), 1 U of DreamTaq DNA Polymerase (Thermo Scientific), and 10 μL of a dilution of bacterial cells in sterile water. The PCR conditions for the confirmation of Pst were as follows: an initial denaturation step of 3 min at 95° C., followed by 35 cycles of 30 s at 95° C., 30 s at 63° C. and 30 s at 72° C. with a final extension step of 10 min at 72° C. [7]. Similarly, PCR conditions for the confirmation of Xeu were as follow: an initial denaturation step of 5 min at 95° C., followed by 35 cycles of 30 s at 95° C., 30 s at 61° C. and 30 s at 72° C. with a final extension step of 10 min at 72° C. [8]. PCR products were separated on 0.8% agarose gels stained with GreenSage Premium (Nzytech, Lisbon, Portugal).

In an embodiment, macroscopic tomography reconstruction is performed by recording the spectra to the whole grape in two different apparatus: i. direct transmittance using the described device; and ii. full transmittance using a power led light source as opposed to the transmittance probe with light collection fibers, using a reflectance probe and HR4000 spectrometer from Ocean Insight. The same grape was afterward separated into its different tissues (skin, pulp, and seeds) for the spectral recording of these grape components. A total of 192 grapes were used to provide statistical support to macroscopic reconstruction.

Leaf samples of each plant were excised of the plant and readily analysed: each leaf was deposited on a single glass slide, supported by a platform featuring a pinhole slit (<0.5 mm) from which white light was projected (>65000 lux). The stainless-steel probe was then placed perpendicularly to the leaf (adaxial side) on select spots (3×) throughout each leaf (radial orientation) for spectral acquisition. This configuration minimises light scattering, allowing light collimation towards the fiber within the probe, maintaining a minimal measurement area (200 μm), and providing maximum discrimination by leaf scanning. No further sample preparation was required nor performed.

FIG. 5 presents an embodiment of a macroscopical setup consisting of a manual probe for data acquisition on select matrix spots.

Spectroscopy measurements were performed using UV-Vis-NIR fiber optic (I.D. 200 μm) stainless steel probe connected to an Ocean Insight model HR4000 spectrometer, with dynamic acquisition times, so that spectral data was fully comprehended within the linear range of the spectrometer. Data acquisition was performed within the 200-1100 nm range, and every 24 h, until the 4th day, and one final sampling moment at 144 h, in a total of 5 sampling moments per group.

Leaf samples of each plant were excised of the plant and readily analysed: samples were laid between glass slides, and placed on the microscopical setup for data acquisition. A 5×5 grid (millimetre spacing) sampling grid was devised on each leaf, comprising of 25 data points of spectroscopic data; sampling grid initial coordinates were randomly set, yet assuring the avoidance of the leaf main stem line. No further sample preparation was required nor performed.

The hyperspectral microscopy system, consisting of a Zeiss Axiovert.A1, in transmission mode, was modified and customised to perform 3D hyperspectral microscopy for sequential x, y, z mapping. The following modifications were performed:
i. Ocular adapter for spectral measurements: an adapter was developed to adapt a fiber optics spectrometer (HR4000) to the ocular. The adapter allows aligning the measurement fiber with the center of the microscope camera, allowing to synchronise the spectral measurement with the recorded image at any magnification and position (x, y, and z-axis);
ii. Adapter for direct in vivo leaf measurements using two glass slides;
iii. Hyperspectral scanning and imaging software: hyperspectral images were performed by linear scanning of the leaf at each z depth, producing a spectral image at each depth. Tomography is recorded layer-by-layer. Imaging software was developed to show each layer using triangular finite elements for allowing continuous rendering of spectral data.

Microscopical imaging was performed using Best Fit microscopy option for macro (Red, Green, Blue—RGB) images, under the same conditions as for microscopical hyperspectral imaging, every 24 h, until the 4^{th} day, and one final sampling moment at 144 h, in a total of 5 sampling moments per group.

FIG. 6 presents an embodiment of a hyperspectral microscope setup composed by a Zeiss Axiovert.A1 10, connected to an Ocean Insight HR4000 spectrometer 30 via an UV-Vis-NIR 200 μm (I.D.) optical fiber 20, fine-tuned for accurate, simultaneous spectral acquisition of a sampled micrograph.

The basis of information relationship within different scales of spectral measurements is performed by hierarchical relationship of super-space and corresponding sub-spaces. The sub-spaces conserve the latent space variables of the different layers of a hyperspectral microscope image, that is, the features of these images are linear combinations of the vectors spawning the sub-space at a given space coordinate [9,10]. These characteristics are used to derive a super-space of characteristics, translating the combination of a particular hyperspectral image or tomography. The super-space characteristics are afterwards possible to be related to the macroscopic feature space to establish the relationship between macroscopic and microscopic measurements.

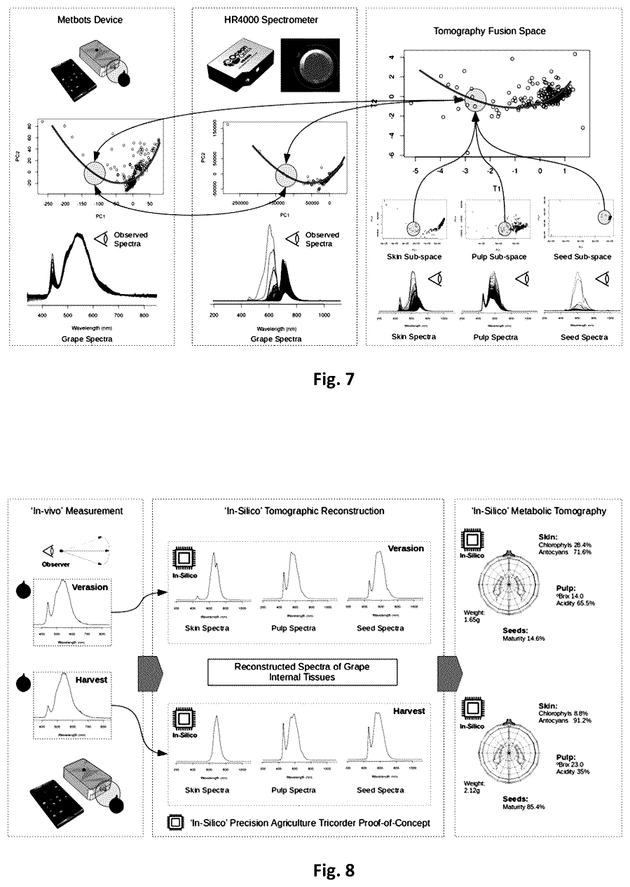

Results of the reconstruction of the macroscopic tomography using hierarchical latent variables are presented in FIG. 7. In this example, we demonstrate that although the observed spectra in different types of equipment, e.g., robotic device and HR4000 spectrometer, are not similar in terms of their shape, these carry the same information. The two signals' variance space is equivalent. Such means that both are observing the same spectral data but with a different type of spectral fingerprint due to the optical configuration. This fingerprint carries the same physical and chemical information about the observed grapes.

Spectra of the grapes along the maturation follow the displayed parabola of FIG. 7, in both variance spaces of the robotic device and HR4000 spectrometer. This parabolic shape is due to the evolution of grape maturation and is also observed in the tomography fusion space, which is obtained by the hierarchical relationship between the latent sub-spaces of the skin, pulp, and seeds tissues spectral data.

The tomographic fusion space is obtained by the information fusion of the variance sub-spaces of spectral characteristics of the skin, pulp, and seeds, obtained by their observed spectra, as seen on FIG. 7. The relationship between the robotic device, HR4000, tomography fusion space and the information of the corresponding tissue is bi-directional, i.e., it possible to infer the tissues spectra from the grape spectra—and vice-versa—to estimate the observed grape spectra from the tissue spectra.

This case-study shows how spectral data is correctly represented by its latent structures, spaces, and sub-spaces. The spectral data in tomography, as well as, between different physical scales, is well represented by a hierarchical relationship of the latent structures from each scale. The use of latent structures is highly computationally efficient, as these methods perform all computations in a compressed space, and therefore, as the computational cost is low, the proposed method is ideal for running in low-cost IoT (Internet of Things) hardware, allowing the development of very cost-effective hardware/software relation for metabolic tomography.

FIG. 8 exemplifies the reconstruction of the skin, pulp, and seed spectra from the grape spectra recorded by the robotic device from two different grapes collected during veraison and near harvest. At the human eye, these grapes look very similar, and the recorded spectra from the robotic device to the untrained eye show low variation. The grapes are however in very distinct development stages, and their tissues have very significant compositional differences. By using the relationship between the robotic variance space and tomography fusion space, the computed spectra from the skin, pulp, and seed spectra exhibit very significant differences typical of veraison and near harvest development stages. These significant differences correspond to compositional differences in skin pigmentation (chlorophylls vs anthocyanins), sugars and acids accumulated in the pulp tissues, and seed ripeness status (proportional to lignification and tannins) with estimates presented in FIG. 8.

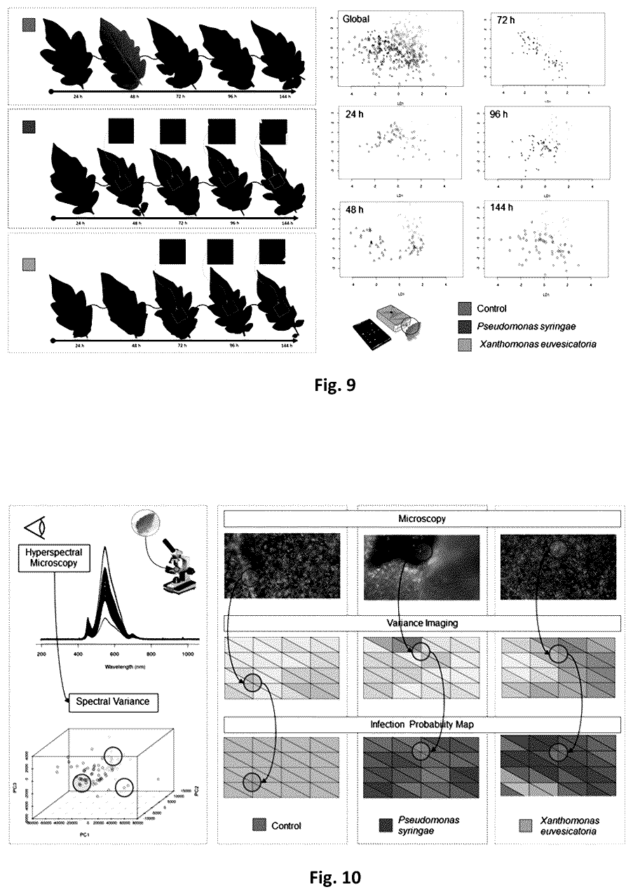

Results of tomato leaf bacterial infection diagnosis by P. syringae and X. euvesicatoria are presented in FIG. 9. These show the infection is extremely aggressive to cause damage to the tomato leaf. Even at the first 24 h, spectral analysis shows that chemical composition is already significantly different between the control, P. syringae and X. euvesicatoria.

At this stage, P. syringae exhibits a higher degree of discriminance than X. euvesicatoria, when compared to the control samples. As the infection progresses, significant differences between the control and infection agent begin to diverge in terms of spectral patterns and corresponding chemical composition, leading to the isolated classifications of each sample type in the linear discriminant analysis presented in FIG. 9. Class separation is obtained for all the sampling times at 24, 48, 72, 96 and 144 hours. Such allows speculating that the infection mechanisms and relationship between an infectious agent and plant response to the infection are characteristic of a particular type of bacteria.

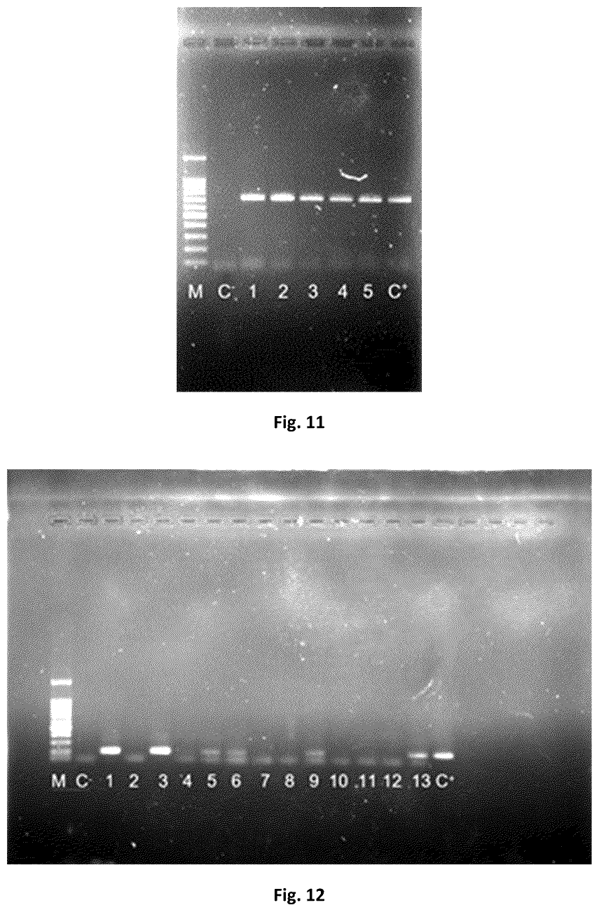

At the naked eye or RGB camera, visible superficial damage appears only after 72 hours after inoculation. Therefore, human or machine vision algorithms (e.g., Deep Learning) will not be able to diagnose the disease before it enters this late stage. Ground truth polymerase chain reaction (PCR) tests shown positive identifications of the presence of P. syringae and X. euvesicatoria after 24 h of inoculation, as shown in FIGS. 11 and 12. The PCR method comprises the amplification of bacterial counts by growth media, during 24 to 48 h at 28° C., not allowing this molecular biology method to be used as rapid response diagnosis. The presented results of the macroscopic tomography case study shows not only a disruptive way of non-invasive disease diagnosis in plants but also the capacity to provide a very early diagnosis.

Results for hyperspectral microscopy tomography are presented in FIG. 10. This figure presents the spectra of hyperspectral tomography of control and infected leaves with P. syringae and X. euvesicatoria at 144 h after inoculation. At this disease stage, spectral variance can be spawned into 3 principal components, which can classify any of the spectra of each category (control, Pst or Xeu). Pseudomonas syringae shows higher degrees of damage, whereas the spectra patterns are consistent with necrosis. Xanthomonas euvesicatoria at this stage has more uniform damage over the hyperspectral data. The spectral patterns from P. syringae or X. euvesicatoria are distinguishable in the 3PC from each other and control samples. It is also shown the estimated probability of infection by each bacteria at each node of the hyperspectral image using a partial least squares discriminant analysis model. Despite the spectral variance being extremely significant at each hyperspectral image, due to both leaf structures and compositional differences, these do not affect classification performance by a linear classifier. For example, the PLS-DA model correctly predicts the control sample with an extremely low probability of infection and extremely high probabilities of infection for P. syringae and X. euvesicatoria. These results are in agreement to the microscopy images, where at 144 h all tissues are infected, not existing non-infected regions. The leaf infected by P. syringae show already advanced necrosis in some spots, and X. euvesicatoria very significant tissue damages. Results also demonstrate that microscopic diagnosis has the potential for early-stage diagnosis, confirming the results obtained by macroscopic tomography.

TABLE 1
Benchmark between PCR, Image analysis and
information according to the disclosure.

| | PCR | Image Analysis | Present disclosure |
|---|---|---|---|
| Type | Genetic | Pattern Recognition | Spectroscopy Analysis |
| Information | Qualitative | Classification | Quantitative and |
| | | | Qualitative |
| In vivo | No | Yes | Yes |
| Metabolites | No | No | Yes |

FIG. 11 presents an embodiment of colony PCR analysis of infected tomato leaves with DNA markers XV14 (713 bp) for the detection of Xanthomonas euvesicatoria LMG 905. C−—Colony PCR using distilled water as template. 1, 2, 3—DNA samples from different bacterial colonies were obtained through a pathogen isolation assay performed 24 h after infection. 4, 5—DNA samples from different bacterial colonies obtained through pathogen isolation assay performed 48 h after infection. C+—Xanthomonas euvesicatoria DNA stored at −80° C. belonging to the laboratorial bacterial collection.

FIG. 12 presents an embodiment of colony PCR analysis of infected tomato leaves with DNA markers PST2 (200 bp) for detection of Pseudomonas syringae pv. tomato. C−—Colony PCR using distilled water as template. 1-6—DNA samples from different bacterial colonies obtained through pathogen isolation assay performed 24 h after infection. 7-13—DNA samples from different bacterial colonies obtained through pathogen isolation assay performed 48 h after infection. C+—Pseudomonas syringae pv. tomato DNA stored at −80° C. belonging to the laboratorial bacterial collection.

To our best knowledge, the state-of-the-art does not provide a tool similar to the present disclosure. Results show that the disclosed technology has high potential to revolutionise precision agriculture in its dimensions by being able to monitor the omics of each plant in vast plantations, providing information for the study of the complex relationship of plant-soil-climate-ecosystem and agricultural practices.

The benchmarks presented in Table 1 show how this type of technology can improve precision agriculture's state of the art. By focusing precision agriculture on plant physiology and its molecular biology, new horizons can be achieved by: i. developing new agricultural practices with omics scientific evidence; ii. introducing a modern way of developing agricultural practices by the use of in silico models from bioinformatics and systems biology; iii. linking plant biotechnology molecular biology in vitro results towards in vivo field applications; and iv. understanding how the physiology of plants inserted into a natural ecosystem responds when compared to laboratory observations.

Flow diagrams of particular embodiments of the presently disclosed methods are depicted in figures. The flow diagrams illustrate the functional information one of ordinary skill in the art requires to perform said methods required in accordance with the present disclosure.

It will be appreciated by those of ordinary skill in the art that unless otherwise indicated herein, the particular sequence of steps described is illustrative only and can be varied without departing from the disclosure. Thus, unless otherwise stated, the steps described are so unordered meaning that, when possible, the steps can be performed in any convenient or desirable order.

It is to be appreciated that certain embodiments of the disclosure as described herein may be incorporated as code (e.g., a software algorithm or program) residing in firmware and/or on computer useable medium having control logic for enabling execution on a computer system having a computer processor, such as any of the servers described herein. Such a computer system typically includes memory storage configured to provide output from execution of the code which configures a processor in accordance with the execution. The code can be arranged as firmware or software and can be organised as a set of modules, including the various modules and algorithms described herein, such as discrete code modules, function calls, procedure calls or objects in an object-oriented programming environment. If implemented using modules, the code can comprise a single module or a plurality of modules that operate in cooperation with one another to configure the machine in which it is executed to perform the associated functions, as described herein.

The disclosure should not be seen in any way restricted to the embodiments described and a person with ordinary skill in the art will foresee many possibilities to modifications thereof. The above-described embodiments are combinable. The following claims further set out particular embodiments of the disclosure.

It is acknowledged the contribution of Fundação Amadeus Dias, Santander Universidades, and the University of Porto in respect of the BIP PROOF 2021 AWARD.

This work was supported by Project “OmicBots: High-Throughput Integrative Omic-Robots Platform for a Generation Physiology-based Precision Viticulture/PTDC/ASP-HOR/1338/2021”, financed by the National Funds through the FCT-Fundação para a Ciência e a Tecnologia, I.P. (Portuguese Foundation for Science and Technology).

### REFERENCES

[1] S. Arshavsky-Graham, E. Segal, Lab-on-a-Chip devices for point-of-care medical diagnostics. Advances in Biochemical Engineering/Biotechnology, Springer, Berlin, Heidelberg, 2020, https://doi.org/10.1007/10_2020_127.
[2] S. Nishat, A. T. Jafry, A. W. Martinez, F. R. Awan, Paper-based microfluidics: simplified fabrication and assay methods, Sens. Actuators B Chem. 336 (2021) 129681, https://doi.org/10.1016/j.snb.2021.129681
[3] T. Zhou, D. Lu, Q. She, C. Chen, J. Chen, Z. Huang, S. Feng, R. You, Y. Lu, Hypersensitive detection of IL-6 on SERS substrate calibrated by dual model, Sens. Actuators B Chem. 336 (2021) 129597, https://doi.org/10.1016/j.snb.2021.129597.
[4] K. Jiang, J. Wu, Y. Qiu, Y. Y. Go, K. Ban, H. J. Park, J. H. Lee, Plasmonic colorimetric PCR for Rapid molecular diagnostic assays, Sens. Actuators B Chem. 337 (2021) 129762, https://doi.org/10.1016/j.snb.2021.129762
[5] J. Prakash, A. Dey, S. Uppal, R. Alexander, A. Kaushal, H. S. Misra, K. Dasgupta, Label-free rapid electrochemical detection of DNA hybridisation using ultrasensitive standalone CNT aerogel biosensor, Biosensors and Bioelectronics, 191 (2021) 113480, https://doi.org/10.1016/j.bios.2021.113480.
[6] J. Liu, X. Wang Plant diseases and pests detection based on deep learning: a review. Plant Methods 17, 22 (2021). https://doi.org/10.1186/s13007-021-00722-9
[7] J. Vieira, M. V. Mendes, P. Albuquerque, P. Moradas-Ferreira, F. Tavares, A novel approach for the identification of bacterial taxa-specific molecular markers. Lett Appl Microbiol. 44 (2007), 506-12. https://doi.org/10.1111/j.1472-765X.2007.02109.x.
[8] P. Albuquerque, C. M. R. Caridade, A. S. Rodrigues, A. R. S. Marcal, J. Cruz, et al., Evolutionary and Experimental Assessment of Novel Markers for Detection of Xanthomonas euvesicatoria in Plant Samples. PLOS ONE (2012) 7, e37836. https://doi.org/10.1371/journal.pone.0037836
[9] J. A. Westerhuis, T. Kourti, and J. F. MacGregor, Analysis of multiblock and hierarchical PCA and PLS models. J. Chemometrics, 12 (1998), 301-321. https://doi.org/10.1002/(SICI) 1099-128X (199809/10) 12: 5<301::AID-CEM515>3.0.CO; 2-S

O. E. de Noord, Theobald E. H., Multilevel component analysis and multilevel PLS of chemical process data. J. Chemometrics, 19 (2005) 301-307. https://doi.org/10.1002/cem.933

J. Chan, S. C. Raju, E. Topol. Towards a tricorder for diagnosing paediatric conditions. The Lancet (British Edition), 394 (2019) 10202, 907-907.

H. Waters, New $10 million X prize launched for tricorder-style medical device. Nature Medicine, 17 (2011) 754-754

1. A computer-implemented method for non-invasive tomographic metabolite characterization of a vegetable or animal sample to be characterized, the sample comprising a plurality of differentiated tissues from a set of previously characterized differentiated tissues, by using a multidimensional latent structure model of differentiated tissues, which was previously obtained by:
scanning tissue samples belonging to each differentiated tissue of the set with a hyperspectral microscope and spectrometer;
acquiring hyperspectral spectrum or spectra for each differentiated tissue sample from the hyperspectral microscope and spectrometer;
analyzing the same tissue samples for the metabolites to be characterized;
calculating the latent structure model from the acquired spectra and analyzed metabolites;
wherein the method comprises the steps of:
spatially scanning the sample to be characterized with a hyperspectral point-of-measurement (POM) optical device and a spectrometer;
acquiring hyperspectral spectrum or spectra from each of a plurality of spatial positions and/or orientations of the POM optical device and spectrometer in respect of the sample to be characterized;
using the previously obtained model to deconvolute the metabolites to be characterized for each of the differentiated tissues from the acquired spectra.

2. The method according to claim 1, wherein the scanning tissue samples belonging to each tissue of the set with a hyperspectral microscope and spectrometer is microscopic, and
the spatially scanning the sample to be characterized with a hyperspectral POM optical device and spectrometer is macroscopic.

3. The method according to claim 2, wherein spatially scanning comprises varying one or more of the following to obtain a spatial diversity of the POM spatial scanning:
displacement of the POM optical device;
point-of-view angle of the POM optical device;
different scales of magnification of the POM optical device; and
moving the POM optical device lines of sight to cause a parallax effect.

4. The method according to claim 1, wherein the multidimensional latent structure model is a hierarchical latent structure model.

5. The method according to claim 4, wherein the hierarchical latent structure model is comprised of a latent variable model comprising a latent variable super-space and corresponding latent variable sub-spaces.

6. The method according to claim 5, wherein the latent variable super-space is a super-space for each of the metabolites to be characterized, further comprising:
translating the combination of hyperspectral data to the metabolites to be characterized;
wherein each of the latent variable sub-spaces is a latent sub-space for spectral data of each of the differentiated tissues.

7. The method according to claim 1, wherein the sample to be characterized is a three-dimensional tissue structure or organ.

8. The method according to claim 1, wherein the metabolites to be characterized comprise pigments, tannins, photosynthesis, energy storage, nutrient uptake, necrosis, infection probability, or combinations thereof.

9. The method according to claim 1, wherein the sample to be characterized is a vegetable sample and the differentiated tissues comprise skin, pulp and seed tissue.

10. The method according to claim 9, wherein the sample to be characterized is a tomato or grape tissue sample.

11. The method according to claim 1, wherein the scanning of tissue samples belonging to each differentiated tissue is carried out along a sampling grid.

12. The method according to claim 1, wherein the scanning of tissue samples belonging to each differentiated tissue is carried out along discrete sampling depths.

13. The method according to claim 1, wherein the spectra are UV-VIS-NIR.

14. A device for non-invasive tomographic metabolite characterization of a vegetable or animal sample to be characterized, the sample comprising a plurality of differentiated tissues from a set of previously characterized differentiated tissues, comprising a computer processor and a non-transitory computer-readable medium comprising a multidimensional latent structure model of differentiated tissues, which was previously obtained by:
scanning tissue samples belonging to each differentiated tissue of the set with a hyperspectral microscope and spectrometer;
acquiring hyperspectral spectrum or spectra for each differentiated tissue sample from the hyperspectral microscope and spectrometer;
analysing the same tissue samples for the metabolites to be characterized; and
calculating the latent structure model from the acquired spectra and analysed metabolites;
wherein the computer processor is configured for:
spatially scanning the sample to be characterized with a hyperspectral point-of-measurement, POM, optical device and a spectrometer;
acquiring hyperspectral spectrum or spectra from each of a plurality of spatial positions and/or orientations of the POM optical device and spectrometer in respect of the sample to be characterized; and
using the previously obtained model to deconvolute the metabolites to be characterized for each of the differentiated tissues from the acquired spectra.

15. A non-transitory computer-readable medium comprising computer program instructions for implementing a device for non-invasive tomographic metabolite characterization of a vegetable or animal sample to be characterized, which when executed by a processor, cause the processor to carry out the method of claim 1.
